(19) United States
(12) Patent Application Publication
(10) Pub. No.: US 2006/0078596 A1
(43) Pub. Date: Apr. 13, 2006

(54) Treatment of muscle fatigue

(76) Inventors: Kieran Clarke, Oxford (GB); Michaela Scheuermann-Freestone, Oxford (GB); Andrew James Murray, Oxford (GB)

Correspondence Address: BELL & ASSOCIATES, 416 FUNSTON ST., SUITE 100, SAN FRANCISCO, CA, 94118, US

(21) Appl. No.: 11/287,803

(22) Filed: Nov. 28, 2005

Related U.S. Application Data
(63) Continuation-in-part of application No. PCT/GB04/02286, filed on May 27, 2004.

(30) Foreign Application Priority Data
Jun. 2, 2003 (GB) GB 0312603.4
Jun. 13, 2003 (GB) GB 0313760.1

(51) Int. Cl. A61K 47/00; A61K 31/60; A61K 31/455; A61K 31/426; A61K 31/192; A61B 5/05

(52) U.S. Cl. 424/439; 514/159; 514/356; 514/369; 514/571; 600/547

(57) ABSTRACT

The present invention involves the use of a compound that reduces the level of free fatty acids circulating in the plasma of a subject in the manufacture of a medicament for the treatment of prevention of muscle (particularly cardiac or skeletal muscle) impairment or fatigue.

The present application is a continuation-in-part of and claims priority to pending International Patent Application Serial Number PCT/GB2004/002286 entitled “Treatment of Muscle Fatigue”, having an international filing date of 27 May, 2004, which in turn claimed priority from Great Britain Patent Application Serial Number GB0312603.4 entitled “Method”, filed 2 Jun. 2003, and Great Britain Patent Application Serial Number GB0313760.1 entitled “Method”, filed 13 Jun. 2003, all of which are herein incorporated by reference in their entirety for all purposes.

### FIELD OF THE INVENTION

This invention relates to the treatment of muscle impairment or fatigue, in particular to the treatment of cardiovascular disease and in particular heart failure.

### BACKGROUND OF THE INVENTION

Cardiovascular disease is the leading cause of death in patients with type 2 diabetes,¹ who have decreased survival after myocardial infarction and increased congestive heart failure and silent ischemia compared with non-diabetic diabetic control subjects.^{1,2} Type 2 diabetes mellitus is a chronic metabolic disorder characterised by insulin resistance, hyperglycemia, hyperinsulinemia and elevated plasma free fatty acids, with poor glycemic control associated with an increased risk of heart failure.^{2,3} Therapeutic interventions that normalise glucose and lipid metabolism reduce the incidence of cardiovascular disease in patients with diabetes,⁴ with metabolic control of diabetes being the most important predictor of cardiovascular morbidity and mortality.¹

In the normal adult heart, free fatty acids, glucose and lactate are metabolised for ATP production in the mitochondria. However, in the diabetic heart, glucose and lactate oxidation are decreased^{5,6} and fatty acid oxidation is increased,⁷ increasing the oxygen requirement per ATP molecule produced.^{2,4,7,8} Positron emission tomographic studies have shown that patients with type 2 diabetes have decreased resting myocardial blood flow rates⁹ and decreased fluorodeoxyglucose uptake rates,¹⁰ yet little is known of cardiac high energy phosphate metabolism in these patients. Similarly, skeletal muscle blood flow,¹¹ and glucose transmembrane transport and oxidation² are decreased in diabetes. Patients with type 2 diabetes have limited exercise tolerance,^{2,12,13} which has been associated with decreased glycemic control¹² and microvascular disease.¹³

WO0/64876 discloses tri-aryl acid derivatives that modulate the function of peroxisome proliferator-activated receptors (PPAR), to decrease triglyceride levels and therefore treat disorders associated with high levels of triglyceride, including diabetes.

WO01/74834 discloses specific compounds that inhibit sodium-dependent glucose transporters and can therefore be used for treating diabetes and associated complications.

WO02/028857 discloses specific compounds that can act as anti-diabetics. The compounds are stated to have multiple activities including the reduction of plasma triglycerides and free-fatty acids.

WO99/24451 discloses adenosine derivatives that inhibit lipolysis and therefore decrease free fatty acid levels.

WO01/51645 discloses polypeptides that decrease free-fatty acid levels, with the purpose of decreasing body mass.

Although the prior art discloses that reduction of free-fatty acid levels is desirable in the treatment or prevention of diabetes and its associated complications, there is no mention that such a reduction in free-fatty acid levels can be used to treat muscle fatigue or impairment.

### SUMMARY OF THE INVENTION

The present invention is based in part on the finding that cardiac high energy phosphate metabolism is significantly altered in patients with type 2 diabetes, despite apparently normal cardiac morphology and function. The alteration in phosphate metabolism correlates with circulating free fatty acid and glucose concentrations. In contrast, skeletal muscle energetics and oxygenation are normal at rest, but deoxygenation and loss of phosphocreatine are faster during exercise and reoxygenation and phosphocreatine recovery are slower following exercise. These findings suggest that alterations in cardiac and skeletal muscle energetics occur early in the pathophysiology of type 2 diabetes and are associated with alterations in metabolic substrates. The findings suggest that lowering free fatty acids will be useful in the treatment of muscle impairment generally, in particular cardiac muscle impairment. Furthermore, the reduction of free fatty acids may be a desirable aim in the treatment of disorders associated with mitochondrial dysfunction. The treatment of cardiac muscle impairment is distinct from the treatment of cardiovascular disease, which is caused by the build up of arthereosclerotic plaques in the vasculature the present invention, by contrast, involves the repair (or prevention of damage to) the cardiac muscle.

In addition, as cardiac muscle energetics and function are strong predictors of mortality and correlate negatively with circulating free fatty acid (FFA) concentrations in patients with heart failure, it has now been realised that increased FFA concentrations, achieved by a high-fat, low-carbohydrate (Atkins) diet, may alter cardiac energetics in healthy subjects and may affect cardiac function.

According to a first aspect of the invention,a compound that reduces the level of free fatty acids circulating in the plasma of a subject is used in the manufacture or prevention of muscle (particularly cardiac or skeletal muscle) impairment or fatigue.

The compound can be used in patients suffering in particular any of the following conditions: diabetes, cardiac impairment, hypopyrexia, hyperthyroidism, metabolic syndrome X, fever, and infection.

The compound can also be used in healthy and/or non-obese subjects.

The compound may be administered in any suitable form and by any suitable route of administration. In one embodiment, the compound may be administered in a food or drink supplement.

In a preferred embodiment, the compound induces mild ketosis. For example, the compound is a ketone body, e.g. a ketone body ester.

In another preferred embodiment, the compound reduces fatty acid levels in blood plasma, e.g. a compound selected form the group consisting of nicotinic acid, salicyclic acid, thiazolidine diones, fibrates, adenosine derivatives, and globular OBG3 polypeptide or fragments thereof.

According to a second aspect of the invention a liquid composition for rehydration during or after exercise comprises water, a sugar carbohydrate and a compound that reduces free fatty acids circulating in the blood plasma.

### DESCRIPTION OF THE DRAWINGS

The present invention is described with reference to the accompanying drawings, wherein:

FIG. 1 shows a typical cardiac ³¹P MR spectra from a normal control (upper spectrum) and a patient with type 2 diabetes (lower spectrum), showing the lower PCr/ATP ratio in the patient; 2,3-DGP (2,3-diphosphoglycerate), PDE (phosphodiesters), PCr (phosphocreatine), α, β, and γ indicate the three phosphate groups of ATP;

FIG. 2 is a graph showing high energy phosphate levels, expressed as the PCr to ATP ratio (Pcr/ATP); trend lines are shown to guide the eye;

FIG. 3 is a graph showing skeletal muscle exercise tolerance, expressed as exercise time, in patients with type 2 diabetes (n=21) and control subjects (n=15); the number of subjects exercising is plotted for each minute of exercise, showing that the patients were unable to exercise for as long as the controls;

FIG. 4 is a graph showing typical calf muscle ³¹P MR spectra from a control subject and a patient with type 2 diabetes at rest (upper panel, number of scans=64), from the same patient at the end of exercise and the same matched control at the equivalent time (5.1 min) of exercise (lower panel, number of scans=16); Pi (inorganic phosphate), PDE (phosphodiesters), PCr (phosphocreatine), α, β, and γ indicate the three phosphate groups of ATP; the cytosolic pH was calculated from the chemical shift of Pi relative to PCr; the abscissa shows the chemical shift in parts per million (ppm);

FIG. 5 shows exercise times correlated with HbA1c levels and with skeletal muscle deoxygenation rates during exercise in patients with type 2 diabetes (n=14) and control subjects (n=12); trend lines are shown to guide the eye.

FIG. 6 shows fasting plasma free fatty acid concentrations after two weeks on a diet and after stopping the diet (upper panel), open circles represent subjects before and at the end of two weeks on the diet (n=19), closed circles represent the subgroup of subjects (n=12) after two weeks on the diet and following two weeks of a normal diet, the larger symbols indicate mean values±SEM (**=p<0.01, ***=p<0.001 vs. pre-diet);

FIG. 7 shows plasma free fatty acid concentrations, cardiac PCr/ATP ratios (middle panel) and respiratory quotient (lower panel) over the five days of the diet, open circles represent subjects before start of the diet, and grey triangles represent subjects during the first 6 days of diet (*=p<0.05, **=p<0.01 vs. pre-diet); and

FIG. 8 shows cardiac PCr/ATP correlated with plasma free fatty acid concentrations (upper panel), and left ventricular peak filling rate correlated with cardiac PCr/ATP (lower panel), open circles represent subjects before diet, closed circles represent subjects two weeks on the diet, and open squares represent subjects two weeks after stopping the diet.

### DESCRIPTION OF THE INVENTION

The term “PCr” used herein refers to phosphocreatine; the term “PDE” refers to phosphodiesters; and the term “ATP” refers to adenosine triphosphate, as will be appreciated by the skilled person.

The present invention shows that high energy phosphate metabolism is significantly impaired in cardiac and skeletal muscle in patients with type 2 diabetes who have apparently normal cardiac morphology and function. The PCr/ATP ratios correlated negatively with the circulating free fatty acids in all subjects tested and positively with the plasma glucose in patients with diabetes. Furthermore, faster skeletal muscle PCr loss is found together with pH decline and deoxygenation during exercise in patients with diabetes and slower PCr recovery following exercise; the PCr recovery half-times correlate with the reoxygenation times for all subjects.

Cardiac Metabolism

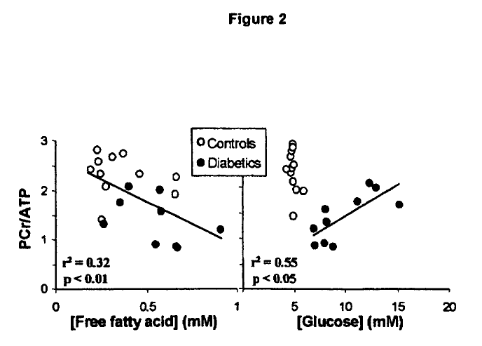

Hyperinsulinemia, hyperglycemia and increased lipid and lipoprotein abnormalities associated with type 2 diabetes may negatively influence myocardial performance,⁴ but, in the early stages of diabetes mellitus, systolic function is often preserved despite changes in cardiac substrate metabolism.^{5,6} It is unknown whether substrate changes in diabetes mellitus^{2,4-7} alter myocardial high energy phosphate metabolism. ³¹P MRS is the only non-invasive tool for measurement of high energy phosphate metabolism in the human heart, although limited to measurement of the PCr/ATP ratio in routine patient studies. Despite the limited information obtainable from human heart, compared with ³¹P MRS of isolated heart⁶ and skeletal muscle;¹⁵ a ³¹P MRS study of patients with dilated cardiomyopathyhas shown a low cardiac PCr/ATP ratio to be a strong predictor of total and cardiovascular mortality, superior to the measurement of ejection fraction.¹⁸ The studies of the present invention revealed that the myocardial PCr/ATP ratio was 35% lower in type 2 diabetic patients, who had normal cardiac function, than in healthy controls. The PCr/ATP ratio correlated negatively with the plasma free fatty acid concentrations in all subjects because free fatty acid concentrations are not under tight metabolic control (FIG. 2). Increased fatty acid availability results in increased free fatty acid uptake and oxidation in the mitochondria,^{2,7} and increased expression of mitochondrial uncoupling proteins,¹⁹ both of which decrease the amount of ATP produced per molecule of oxygen consumed in the mitochondrial electron transport chain.^{2,19} Therefore the diabetic heart has an increased requirement for oxygen.⁸

The hyperglycemia that occurs with diabetes is known to compensate for the impaired capacity for myocardial glucose transport.⁷ Glucose uptake is important for glycolytic ATP production during ischemia, low glucose uptake increasing ischemic injury in the heart.⁶ Patients with type 2 diabetes have decreased fluorodeoxyglucose uptake rates,¹⁰ decreased resting myocardial blood flows⁹ and an increased incidence of silent ischemia.¹ In the study detailed below, the lower cardiac PCr/ATP ratios in the patients who had lower plasma glucose concentrations suggested that decreased glucose availability may have limited glucose uptake. Although plasma lactate levels were 40% higher in the diabetic patients, and lactate is a metabolic substrate for the heart, the lack of a correlation between lactate levels and the cardiac PCr/ATP ratio was possibly because lactate oxidation is inhibited more than glucose oxidation in the diabetic heart.²⁰

Skeletal Muscle Metabolism

Although cardiac high energy phosphate metabolism was abnormal in the patients with diabetes, the results of the study found that skeletal muscle energetics, pH and oxygenation were normal at rest. All subjects fatigued after the same tissue deoxygenation and with the same loss of PCr, increase in free ADP and at the same acidic pH. This suggests that substrate availability or metabolism and glycogen levels were not limiting the skeletal muscle energetic changes. Additionally, faster loss of PCr and decrease in pH during exercise with slower PCr recovery was found after exercise in the patients with diabetes. The PCr recovery half-times correlated with the plasma HbA,c and glucose, but not with fatty acid or lactate concentrations. However, deoxygenation was faster during exercise in the patients with diabetes and reoxygenation was slower following exercise and correlated with the PCr recovery half-times, suggesting that tissue oxygen availability was limiting ATP production. Elevated levels of HbA_{1c} have been associated with microvascular complications^{3,21,22} and reduced exercise capacity.¹² In the study indicated below, the exercise times and the reoxygenation times correlated with the HbA_{1c} levels, indicating that abnormal skeletal muscle oxygenation in the patients with diabetes may have been related to microvascular disease. In diabetic patients with intermittent claudication, skeletal muscle reoxygenation took ˜4 times longer than in normal subjects and provided a more sensitive measure of lower leg claudication than ankle pressure measurements.²³ Consequently, microvascular disease may explain most, if not all, of the abnormalities in skeletal muscle high energy phosphate metabolism that were observed in patients with diabetes.

The following is a non-exhaustive list of conditions that are associated with high levels of free fatty acids. Patients with the following disorders may benefit from treatment with compounds that reduce the levels of free fatty acids: neurodegenerative diseases including, but not limited to, Alzheimer's disease, Parkinson's disease, Huntington's chorea; hypoxic states including, but not limited to, angina pectoris, extreme physical exertion, intermittent claudication, hypoxia, stroke, myocardial infarction; insulin resistant states including, but not limited to, infection, stress, obesity, diabetes, heart failure; and inflammatory states including, but not limited to, infection, autoimmune disease. Further studies demonstrated a positive correlation between circulating free fatty acids (FFAs) and the levels of the mitochondrial uncoupling proteins UCP-2 (r=0.42;P<0.001) and UCP-3 (r²=0.22; P<0.05) but no correlation was found with any other plasma metabolite. This suggests that increased circulating FFA in humans increase UCP expression and thereby decrease cardiac efficiency. Reducing circulating FFA levels in patients may therefore provide a new treatment for heart failure that increases the efficiency of cardiac hydraulic work. The following is a non-exhaustive list of conditions that are associated with mitochondrial dysfunction. Patients with the following disorders may also benefit from treatment with compounds that reduce the levels of free fatty acids in the blood:

Essential hypertension

Cardiomyopathy

Congenital muscular dystrophy

Immune (Hyper Thyroid)

Fatigue & Exercise intolerance

Hypertension

Kidney disease

Longevity (Aging)

MELASL (Mitochondrial Encephalomopathy, Lactic Acidosis, and Stroke-Like episodes)

Deafness

Multiple symmetric lipomatosis

Myalgias

Myoglobinuria

Myopathy syndromes

Neoplasms (Cancer)

Optic atrophy

Rhabdomyolysis:mtDNA

Sudden infant death (SIDS)

Wilson's disease

The person skilled in the art will recognise that there are many compounds that are known to reduce fatty acid levels in blood plasma. For example, the compounds disclosed in the international (PCT) patent publications WO-A-00/64876, WO-A-01/74834,WO-A-02/028857,WO-99/24451, and WO-01/51645 (the content of each being incorporated herein by reference in their entirety) may be used in the present invention. Suitable compounds result in decreased free fatty acid levels of at least 5%, more preferably at least 20%, and most preferably at least 30%.

The medicament may be prepared in any convenient formulation, for oral, mucosal, pulmonary, intravenous or other delivery form. Suitable amounts will be apparent to the person skilled in the art, depending on the severity of the condition to be treated, age and weight of the patient, as will be appreciated by the skilled person.

In one aspect of the invention, there is a composition for rehydration during or after exercise, the composition comprising water, a sugar carbohydrate and a compound that reduces the levels fo free fatty acids in the plasma of a patient. The composition may also comprise suitable flavourings, colourants and preservatives, as will be appreciated by the skilled person. The carbohydrate sugar is present as an energy source, and suitable sugars are known, including glucose and trehalose.

According to a separate aspect there is a method for the treatment or prevention of muscle, particularly cardiac or skeletal muscle, impairment or fatigue or mitochondrial dysfunction. The method is carried out by administering a compound that reduces the level of free fatty acid in the plasma of a patient. The compound will usually be administered in an amount to achieve a circulating free fatty acid concentration of less than 0.5 mM.

In addition to studying the effects of free fatty acid levels on cardiac muscle function, in patients with type 11 diabetes, it was also shown that increased circulating free fatty acid concentrations, resulting from a two week, high-fat, high-protein, low-carbohydrate (Atkins) diet, are associated with impaired cardiac energetics and function. The negative correlations between free fatty acid concentrations and cardiac energetics and the positive correlation between cardiac energetics and diastolic function, suggest that circulating free fatty acids alter cardiac energetics, which, in turn, may impair cardiac function.

The healthy adult heart utilises free fatty acids, glucose and lactate to generate ATP via mitochondrial oxidation. The availability, uptake and metabolism of these substrates varies with altered cardiac perfusion and function,²⁶ in metabolic diseases,²⁷ and with changes of diet.^{28,29} We found negative correlations between cardiac energetics and circulating free fatty acid concentrations in patients with heart failure³⁰ and in patients with type 2 diabetes mellitus with preserved cardiac function.³¹ These studies suggested that increased metabolism of free fatty acids may alter cardiac energetics, and that abnormal cardiac energetics may precede cardiac dysfunction. The high-fat, high-protein, low-carbohydrate (Atkins) diet raises free fatty acid concentrations and caused dyotolic dysfunction in normal subjects. Here, two weeks of high-fat, low-carbohydrate diet almost doubled circulating free fatty acid and 3-β-hydroxybutyrate concentrations, whereas glucose, insulin, insulin resistance and triglyceride concentrations decreased. Associated with the increased plasma free fatty acid concentrations were lower cardiac PCr/ATP ratios. This effect was observed after one day of diet to continue throughout the two weeks of diet, and was accompanied by a reduction in respiratory quotient, an index of the ratio of fat to carbohydrate oxidation,²⁵ indicating increased free fatty acid oxidation. These effects reversed within two weeks of returning to a normal diet.

Measurement of free fatty acids can be accomplished by methods known in the art, and disclosed herein.

Having appreciated the importance of measuring FFA levels in serum, it may also be desirable for individuals to maintain a regular watch on their FFA levels, in particular if the individual is on a high-fat low-carbohydrate diet or suffers cardiac dysfunction or diabetes. It will therefore be of benefit to have a portable device for measuring FFA levels in serum.

Many different devices for measuring FFA levels in serum are within the scope of the present invention. Devices may be constructed to measure FFA levels using the fluorescence probe ADIFAB (acrylodated intestinal fatty acid binding protein) as disclosed in Richieri et al, J. Lipid Res.,1995; 36(2): 229-40, the content of which is incorporated herein by reference.

The device will usually be a hand-held device and will contain, either in the device or in kit form, all the components and reagents necessary to allow the measurement to be made.

There are now many hand-held devices available commercially which can be adapted for the purpose of measuring FFA in a serum sample.

Devices based on micro electrodes are particularly suitable. For example, International patent publication numbers WO-A-03/097860, WO-A-03/012417 and WO-A-03/056319 (the content of each of which is incorporated herein by reference) disclose “biosensor” devices for measuring biological reactions using micro electrodes. The micro electrodes comprise typically an electrochemical cell which, either alone or in combination with a substrate onto which it is placed, is in the form of a receptacle. The cell comprises a counter electrode and a working electrode with the working electrode being in a wall of the receptacle. The electrochemical cell will also comprise an electro-active substance which causes an electrochemical reaction when it comes into contact with the free fatty acids. For example, the electrode-active substance may be the enzyme acyl-CoA synthetase.

The device may also be based on a colourimetric system. For example, Tinnikor et al., Clin. Chim. Acta., 1999; 281: 159, discloses a colourimetric system for measuring free fatty acids based on fatty acid-Cu complexes. The content of this disclosure is contained herein by reference.

The following examples illustrate the invention with reference to the accompanying figures.

### EXAMPLE 1

Subjects and Protocol

Patients with type 2 diabetes (n=21) aged between 18 and 75 years with no evidence of cardiovascular disease or ECG-detectable evidence of ischemia were included in this study. Five patients were diet-controlled only, 6 patients each were treated with either a sulfonylurea drug or metformin, and 4 patients were treated with metformin and a sulfonylurea. Patients on insulin therapy were excluded. Patients were matched for age, sex and body mass index with healthy control subjects (n=15).

All procedures were conducted on the same day, at the same time of day, for each subject. Subjects were fasted overnight for 12 h before blood sampling and echocardiography. After a small breakfast, the cardiac (rest) and skeletal muscle (exercise) magnetic resonance spectroscopy (MRS) protocols were performed and the subjects had lunch. For the near-infrared spectrophotometry (NIRS) measurements of muscle oxygenation, the MRS exercise protocol was repeated outside the magnet because the NIRS probe was magnetic. The MRS and NIRS exercise bouts were separated by two hours of ambulatory rest and 30 minutes of supine rest, to ensure that all variables were stable.

Blood Tests and Echo cardiography

Fasting blood was taken to determine glucose and glycosylated hemoglobin (HbA₁c) levels, lipid profiles and free fatty acid levels (Wako NEFA C enzyme assay, Wako Chemicals, Neuss, Germany). Because our magnetic resonance (MR) scanner was capable of MR spectroscopy, but not of precise left ventricular function analysis, we assessed cardiac function using a SONOS 5500 echocardiography machine (Hewlett Packard, Bracknell, UK). Left ventricular dimensions and mass index were obtained using M-mode echocardiography, and ejection fraction was calculated from left ventricular volumes, derived using the modified Simpson's rule. Diastolic function (early flow velocity (E) and late atrial contraction (A); E:A) was evaluated by acquisition of a pulsed Doppler recording trace through the mitral valve, with the sample volume positioned just above the mitral valve leaflet tips.

Measurements of Cardiac Muscle Metabolism

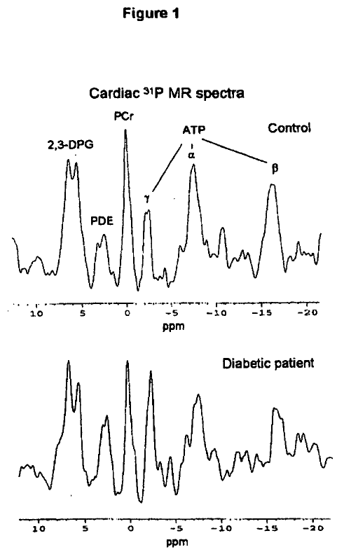

Cardiac high energy phosphate metabolism was measured using ³¹P MRS on a 2 Tesla whole-body magnet (Oxford Magnet Technology, Eynsham, UK) which was interfaced to a Bruker Advance spectrometer (Bruker Medical GmbH, Ettlingen, Germany). Cardiac ³¹P MRS was performed with the subject in the prone position, as previously described.¹⁴ Briefly, subjects were positioned with their heart at the isocentre of the magnet, which was confirmed using standard multislice spin-echo proton (¹H) images acquired with a double-rectangular surface coil placed around the chest (relaxation time TR=heart rate, echo time TE=25 ms, slice=10 mm, 15 mm spacing). Once the position was verified, the coil was exchanged for a circular proton surface coil (diameter 15 cm), and shimming was performed to optimise the magnetic field homogeneity over the heart. Finally, a ³¹P surface coil (diameter 8 cm) was used to acquire cardiac spectra using a slice-selective, one-dimensional chemical shift imaging (1 D-CSI) sequence, including spatial presaturation of lateral muscle (FIG. 1). An 8 cm thick transverse slice was then excited, followed by one-dimensional phase encoding into the chest to subdivide signals into 64 coronal layers, each 1 cm thick (TR=heart rate, 16 averages). All ¹H and ³¹P spectral acquisitions were cardiac gated and saturation corrected. Spectra were Fourier-transformed, and a 15 Hz line broadening was applied. Spectra were fitted using a purpose-designed interactive frequency domain-fitting program. After fitting, the ATP peak area was corrected for blood contamination according to the amplitude of the 2,3-diphosphoglycerate (2,3-DPG) peak and the phosphocreatine (PCr)/ATP and phosphodiester (PDE)/ATP ratios were calculated and corrected for saturation as described earlier.14 The chemical shift differences between the _- and _-phosphate phosphate peaks of ATP were used as a measure of intracellular free magnesium concentrations.

Measurements of Skeletal Muscle Metabolism

³¹P MRS of the right gastrocnemius muscle was performed using the 2 Tesla magnet (see above) with the subject in a supine position and a 6 cm diameter surface coil under the muscle, as previously described.¹⁵ Spectra were acquired using a 2 s interpulse delay at rest (64 scans/spectrum) and during exercise and recovery (16 scans/spectrum).¹⁵ The muscle was exercised by plantar flexion against a standardised weight (10% lean body mass) at 0.5 Hz through a distance of 7 cm, with subsequent further increases of weight (2% of lean body mass every minute), and subjects were exercised until fatigued. Relative concentrations of inorganic phosphate, PCr and ATP were obtained using a time-domain fitting routine (VARPRO, R. de Beer, Delft, Netherlands) and were corrected for partial saturation. Absolute concentrations were obtained assuming that the concentration of cytosolic ATP was 8.2 mmol.l⁻¹ intracellular water and intracellular pH was calculated from the chemical shift of the Pi peak relative to PCr (δPi; measured in parts per million, ppm), using the equation:
pH=6.75+log(δPi−3.27/5.69−δPi)
The chemical shift differences between the α- and β-phosphate peaks of ATP were used as a measure of intracellular free magnesium concentrations. Free cytosolic [ADP] was calculated from pH and [PCr] using a creatine kinase equilibrium constant¹⁶ (K_{ck}) of 1.66×10⁹.M⁻¹ and assuming a normal total creatine content of 42.5 mmol.l⁻¹, using the equation:
[ADP]=[ATP][total creatine]/[PCr][H⁺]K_{ck}

At the end of exercise, because glycogenolysis had stopped and PCr resynthesis was purely oxidative, analysis of PCr recovery provided information about mitochondrial function. Recovery half-times for PCr and ADP, and initial rates of PCr recovery, were calculated as previously described.¹⁵

Measurements of Skeletal Muscle Oxygenation

Muscle oxygen saturation (SmO₂) was measured using dual-wavelength NIRS (INVOS 4100 Oximeter, Somanetics, Troy, USA), with the light emittor and two sensors placed over the medial head of the right gastrocnemius muscle.¹⁷ SmO₂ was determined using the ratio of absorbance at the wavelengths of 733 nm and 809 nm, which estimated deoxygenated and the sum of deoxygenated and oxygenated hemoglobin, respectively. SmO₂ was measured in deep tissue, predominantly at a depth of 2 cm, this being dependent on differentiating between absorption at the interoptode distances of 3 and 4 cm. As determined by such spatial resolution, the SmO₂ was little, if at all, influenced by cutaneous and subcutaneous blood flow.¹⁷ In muscle, ˜75% of blood is in venules or veins, and the INVOS 4100 spectrophotometer has been calibrated against a tissue oxygen saturation in arterial (25% of the signal) and internal jugular vein (75% of the signal) blood. With spatially resolved dual-wavelength NIRS of skeletal muscle, 100% saturation refers to total oxygenation of hemoglobin and myoglobin, as myoglobin attenuates near-infrared light with an absorption spectrum comparable with that of hemoglobin. The muscle NIRS measurements were made in 12 control subjects and in 14 patients with type 2 diabetes.

Statistical Analysis

Data analysis comparing patients with type 2 diabetes and control subjects was performed using the Student's t test and correlations between data sets were determined using the Pearson correlation coefficient. Data are presented as means±standard error of the mean (SEM). Statistical significance was taken at p<0.05.

Results

Patient Characteristics and Echocardiography Results

There were no significant differences in sex, age or body mass index between the patients with type 2 diabetes and the control subjects (Table 1).

TABLE 1
Patient characteristics and echocardiography parameters, and
fasting blood metabolite concentrations, in control subjects (n = 15)
and patients with type 2 diabetes (n = 21).

| | Control subjects | Diabetic patients |
|---|---|---|
| Number of males (% of n) | 11(73%) | 15(71%) |
| Age (y) | 52 ± 3 | 57 ± 2 |
| Body mass index (BMI, kg · m⁻²) | 25.2 ± 0.4 | 28.6 ± 0.5 |
| Diabetes duration (y) | — | 3.3 ± 0.6 |
| Systolic blood pressure (mmHg) | 126 ± 5 | 133 ± 3 |
| Diastolic blood pressure (mmHg) | 76 ± 2 | 74 ± 2 |
| Mean heart rate (beats · min⁻¹) | 70 ± 5 | 68 ± 2 |
| LVESD (cm) | 2.8 ± 0.2 | 3.0 ± 0.2 |
| LVEDD (cm) | 4.3 ± 0.2 | 4.9 ± 0.2 |
| IVSD (cm) | 0.8 ± 0.1 | 1.1 ± 0.1 |
| LVMI (g · m⁻²) | 141 ± 19 | 141 ± 10 |
| E/A | 1.29 ± 0.05 | 0.98 ± 0.12 |
| EF | 0.60 ± 0.02 | 0.61 ± 0.06 |
| HbA₁c (%) | 5.7 ± 0.1 | 8.3 ± 0.4*** |
| Glucose (mmol · l⁻¹) | 5.1 ± 0.1 | 9.5 ± 0.6*** |
| Free fatty acids (mmol · l⁻¹) | 0.40 ± 0.05 | 0.55 ± 0.04* |
| Lactate (mmol · l⁻¹) | 1.1 ± 0.2 | 1.5 ± 0.1* |
| Cholesterol (mmol · l⁻¹) | 4.9 ± 0.2 | 4.7 ± 0.3 |
| Triglycerides (mmol · l⁻¹) | 1.4 ± 0.2 | 1.8 ± 0.2 |
| HDL cholesterol (mmol · l⁻¹) | 1.3 ± 0.1 | 1.1 ± 0.1 |

Data are expressed as means±SEM. LVESD, left ventricular end-systolic diameter; LVEDD, left ventricular end-diastolic diameter; IVSD, interventricular septum diameter; LVMI, left ventricular mass index; E/A, early flow velocity to late atrial contraction ratio; EF, ejection fraction. HbA₁c, glycosylated hemoglobin; HDL, high density lipoprotein. *, p<0.05; ***, p<0.001 vs. control. Mean duration of type 2 diabetes was 3.3±0.6 years from the time of diagnosis. Systolic and diastolic blood pressures and heart rates were similar in the two groups. Echocardiography showed normal left ventricular systolic and diastolic function in patients with no abnormalities in left ventricular chamber thickness or diameter, or any other parameter (Table 1). The patients with diabetes had no history of cardiovascular disease and no clinical signs of impaired cardiac or skeletal muscle blood flow.
Blood Parameters

Fasting blood HbA₁c and glucose levels were 1.5-fold and 1.9-fold higher, respectively, in patients with type 2 diabetes than in controls (Table 1). Plasma levels of free fatty acids were 1.4-fold higher in diabetic patients, as were lactate levels. Total cholesterol, triglycerides, and HDL cholesterol were normal in the patients with diabetes.

Cardiac High Energy Phosphate Metabolism

FIG. 1 shows typical examples of cardiac ³¹P MR spectra from a normal subject (PCr/ATP=2.35) and a patient with type 2 diabetes (PCr/ATP=1.35). The mean cardiac PCr/ATP ratio was 2.30±0.12 in control subjects, but was decreased by 35%, to 1.50±0.11 (p<0.001), in patients with diabetes. The PCr/ATP ratios correlated negatively with the plasma free fatty acid concentrations in all subjects (r²=0.32; p<0.01; FIG. 2), and positively with fasting plasma glucose concentrations in the diabetic patients (r²=0.55; p<0.05; FIG. 2), but there were no correlations with plasma lactate or HbA_{1c} levels. The PDE/ATP ratios were the same in the controls (0.51±0.06) and the diabetic patients (0.51±0.12), as were the chemical shift differences between the α- and β-phosphate peaks of ATP, being 8.3±0.4 and 8.5±0.1 ppm for controls and diabetic patients, respectively.

Skeletal Muscle High Energy Phosphate Metabolism

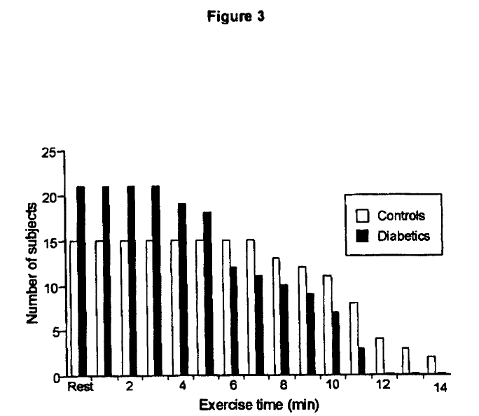

We found that the average exercise times for the patients with diabetes were 32% shorter, at 7 min, compared with the control subjects at 11 min (Table 2 and FIG. 3).

TABLE 2
Skeletal muscle energy metabolites, pH and oxygenation at rest,
during exercise and at the end of exercise in control subjects and
patients with type 2 diabetes.

| | Control subjects | | Diabetic patients | |
|---|---|---|---|---|
| | Rest | End-exercise | Rest | End-exercise |
| PCr | 34 ± 1 | 17 ± 2 | 35 ± 1 | 18 ± 2 |
| (mmol · l⁻¹) | | | | |
| Pi (mmol · l⁻¹) | 4.6 ± 0.2 | — | 4.7 ± 0.1 | — |
| pH | 7.04 ± 0.01 | 6.90 ± 0.04 | 7.04 ± 0.01 | 6.84 ± 0.05 |
| ADP | 15 ± 2 | 60 ± 6 | 11 ± 1 | 57 ± 8 |
| (μmol · l⁻¹) | | | | |
| δ(α-β | 8.29 ± 0.01 | 8.31 ± 0.07 | 8.28 ± 0.01 | 8.40 ± 0.07 |
| ATP (ppm) | | | | |
| Oxygen | 68 ± 3 | 57 ± 3 | 71 ± 2 | 60 ± 3 |
| saturation (%) | | | | |

| | Control | |
|---|---|---|
| | subjects | Diabetic patients |
| | Exercise | |
| Exercise times (min) | 10.5 ± 0.6 | 7.1 ± 0.6*** |
| PCr hydrolysis (mmol · l⁻¹ · min⁻¹) | 1.6 ± 0.1 | 3.2 ± 0.6* |
| pH decline (pH units · min⁻¹) | 0.013 ± 0.003 | 0.036 ± 0.009* |
| Free ADP production | 4.2 ± 0.5 | 9.5 ± 3.4 |
| (μmol · l⁻¹ · min⁻¹) | | |
| Tissue deoxygenation (% · min⁻¹) | 0.8 ± 0.3 | 2.5 ± 0.4** |
| | Recovery | |
| Initial PCr formation | 20 ± 2 | 15 ± 2* |
| (mmol · l⁻¹ · min⁻¹) | | |
| PCr recovery half-time (s) | 32 ± 3 | 52 ± 7* |
| Free ADP recovery half-times (s) | 18 ± 6 | 19 ± 2 |
| Reoxygenation time (s) | 56 ± 10 | 140 ± 18*** |

Data are expressed as means±SEM. ADP, adenosine diphosphate; PCr, phosphocreatine; Pi, inorganic phosphate; δ(α-P)ATP, chemical shift differences between the α- and β-phosphate peaks of ATP. *, p<0.05; *, p<0.01; ***, p<0.001 vs. control.

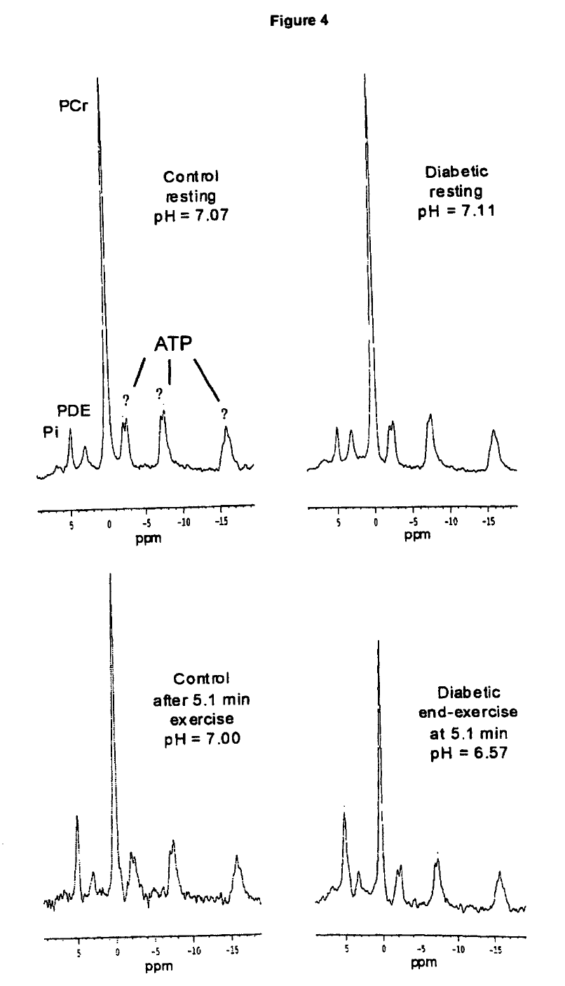

FIG. 4 shows examples of skeletal muscle spectra before and at the end of the standardised exercise protocol in a patient with type 2 diabetes and at the equivalent time (5.1 min) of exercise in a control subject. Under resting conditions, skeletal muscle pH and PCr (PCr/ATP), free ADP and inorganic phosphate concentrations were the same in controls and patients with type 2 diabetes (Table 2). During exercise, PCr hydrolysis was 2-fold faster and the pH decrease was 3-fold faster in the patients with diabetes compared with the control subjects, but the free ADP production rates were not significantly different (Table 2). In all subjects, fatigue occurred when PCr depletion was ˜50% (50±4% in controls vs. 51±4% in diabetics) and at the same pH and free ADP concentrations (Table 2). The free magnesium concentrations remained unaltered during exercise in all subjects (Table 2). Following exercise, the initial rate of PCr recovery was 25% slower and the PCr recovery half-times were 1.6-fold longer in patients with type 2 diabetes than in controls, but the free ADP recovery half-times were the same (Table 2).

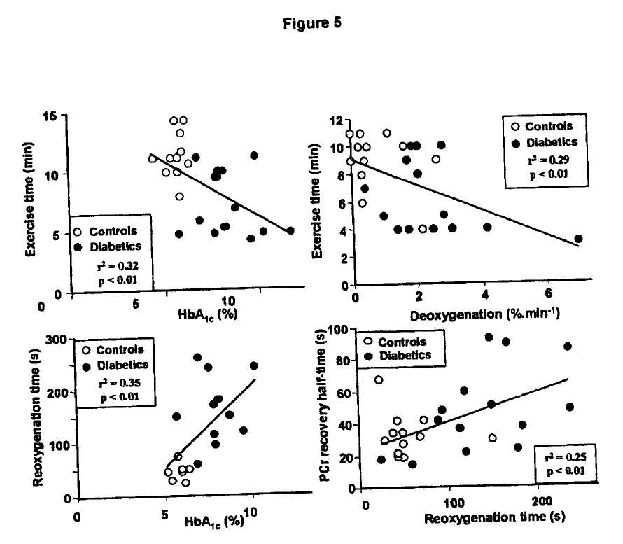

The exercise times correlated negatively with the HbA_{1c} levels (r²=0.32; p<0.01; FIG. 5) and the plasma glucose levels (r²=0.23; p<0.01; correlation not shown), but there were no correlations with the plasma free fatty acid or lactate levels. The rates of PCr hydrolysis and pH decrease during exercise did not correlate with any of the fasting metabolite concentrations. However, the PCr recovery half-times correlated positively with the HbA_{1c} levels (r²=0.40; p<0.001; correlation not shown) and the plasma glucose concentrations (r²=0.16; p<0.05; correlation not shown) for all subjects, but there were no correlations with the plasma free fatty acid or lactate concentrations.

Skeletal Muscle Oxygenation

At rest, gastrocnemius muscle oxygen saturation was stable and the same for both groups, 68% in controls and 71% in diabetics, and all subjects stopped exercising after an 11% decrease in tissue oxygenation measured using NIRS (Table 2). The first diabetic patient stopped exercising after 3 min (FIG. 4), therefore, during the first 3 min of exercise, the rate of deoxygenation was 3.1-fold faster in the type 2 diabetic patients than in the controls (Table 2), and correlated with exercise time (r²=0.29, p<0.01, FIG. 5). Similarly, the reoxygenation times during recovery after exercise were 2.5 times longer in patients with diabetes compared with controls (Table 2), correlating with the HbA_{1c} levels (r²=0.35; p<0.01; FIG. 5) and with PCr recovery half-times (r²=0.25; p<0.01; FIG. 5) in all subjects, but not with the plasma free fatty acid or lactate levels.

The above results show that increases in free fatty acids, associated with type 2 diabetes can contribute to muscle impairment, particularly cardiac muscle impairment. These findings are also relevant to other disorders/conditions associated with high levels of free fatty acids, and so reduction of free fatty acids may be a general aim in reducing the likelihood of muscle impairment, e.g. heart failure.

### Example 2

In a further experiment, cardiac energetics, (phosphocreatine (PCr)/ATP ratios), and function were assessed using magnetic resonance (MR) spectroscopy and imaging, respectively, in 19 healthy subjects before and after two weeks on a high-fat, low-carbohydrate diet and two weeks after returning to their normal diet. The intention was to study whether a high-fat, low-carbohydrate diet alters cardiac energetics in healthy subjects.

Methods

Subjects and Protocol

Nineteen healthy, non-obese subjects volunteered to undergo a high-fat, low-carbohydrate diet for two weeks. Of these 19 subjects, 12 were also studied two weeks after stopping the diet, to determine reversibility of any dietary effects. In another subgroup of 6 subjects, plasma metabolites, cardiac energetics and respiratory quotients were measured daily during the first week of the diet. Subjects fasted for 12 hours (overnight) before samples of blood were taken and cardiac MR measurements (see later) were performed. All tests were conducted at the same time of day. The local Oxford Ethics Committee approved all protocols, and subjects gave their informed consent.

Blood Tests

Fasting blood samples were taken for the measurement of glucose, glycosylated haemoglobin (HbA_{1c}), haematocrit, lipids, 3-β-hydroxybutyrate, insulin (Mercodia AB Insulin ELISA, Uppsala, Sweden) and free fatty acid concentrations (FFA, Wako NEFA C enzyme assay, Wako Chemicals). Relative insulin resistance was calculated using the homeostasis model assessment (HOMA).²⁴ We also measured plasma tumour necrosis factor-a, interleukin 6 (TNF-a, IL-6; Bender MedSystems ELISA, Vienna, Austria) and C-reactive protein concentrations (CRP; ICN Pharmaceuticals ELISA, Orangeburg, USA).

Measurement of Cardiac High-Energy Phosphate Metabolism

Cardiac energy metabolism was assessed with each subject lying in a prone position in a 1.5T clinical MR scanner (Siemens Sonata), using a commercially available heart/liver ³¹Phosphorus/¹H coil (Siemens Medical Systems, Erlangen, Germany), which was positioned under the heart. All acquisitions were cardiac-gated. Subjects were positioned with their hearts in the isocentre of the magnet, confirmed using a stack of standard proton scout images. A series of 32 short axis slices (TrueFisp, 8 mm thick, matrix size 128×96) was acquired, and cardiac ³¹Phosphorus (³¹P) MR spectroscopy was performed using 3D acquisition-weighted chemical shift imaging in the same position. Cardiac PCr/ATP ratios were calculated from voxels placed within the anterior septum using commercially available spectroscopy software (Matlab, MathWorks Inc., Maryland, USA, and were corrected for blood contamination and T1 effects.

Measurement of Cardiac Volumes and Function

Cardiac volumes and function were assessed using cardiac magnetic resonance imaging (MRI) in the 1.5T clinical MR scanner (see above) using steady-state free precession cine images (TE/TR 1.5/3.0 ms, flip angle 60°) with cardiac gating and breath-hold, the patient lying in a supine position. Images were acquired in the two long cardiac axes and in a stack of short axes, spanning the left ventricle consecutively from the base to the apex in 1 cm thick slices.

The short axis slices were analysed using dedicated software (Argus version 2000B, Siemens), and left ventricular volume, stroke volume, cardiac output, ejection fraction and mass index were calculated. Additionally, peak filling rate and peak ejection rate were determined using FLASH cine images of a midventricular short axis slice⁸ for diastolic filling and left ventricular ejection processes, respectively.

Measurement of Respiratory Quotient

To measure fasting respiratory quotients, subjects were seated comfortably in a chair, breathing through a flexible rubber mouthpiece with their nose occluded. Respiratory volumes and flow were measured continuously at the mouth, and gases were analysed by mass spectrometry (Airspec, QP9000, UK) for P_{CO2} and P_{O2} Oxygen consumption and CO₂ production were measured breath-by-breath, and time-weighted averages calculated for each over a 10-minute period of stable breathing. Respiratory quotient was then obtained by dividing average CO₂ production by average O₂ consumption.

Statistical Analysis

Results were analysed using a 2-way ANOVA followed by a modified Student's t-test, and correlations between data sets were determined using the Pearson correlation coefficient. Data are presented as means±standard error of the mean (SEM). Statistical significance was taken at p<0.05.

Results

Subject Characteristics and Blood Metabolite Concentrations

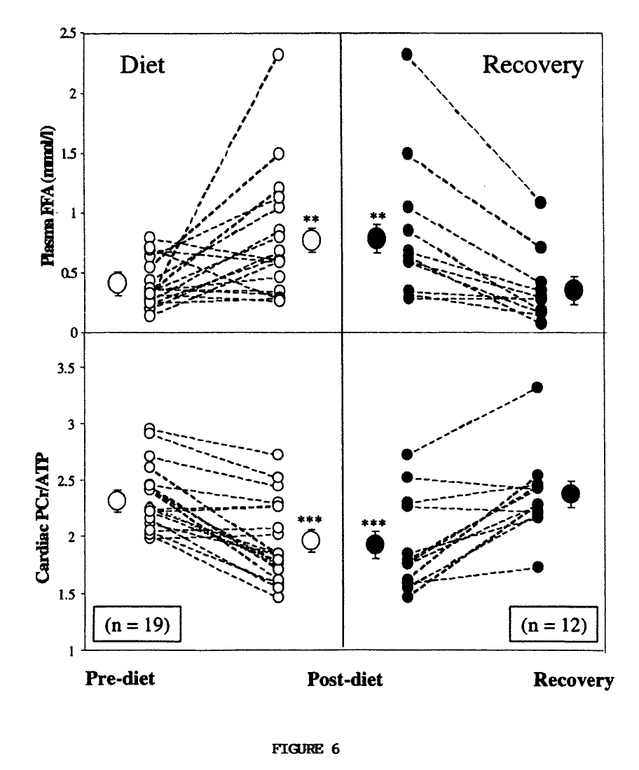

The mean subject age was 36±3 years, and starting body weights and body mass indices were 78±4 kg and 26.3±0.9 kg/m², respectively (Table 3). Two weeks of high-fat, low-carbohydrate diet resulted in a 3.1±SEM kg body weight loss, which was 4±SEM % of the starting body weights, and a BMI decrease of 1.1±SEM kg/M². During the two weeks of the diet, fasting plasma free fatty acid concentrations increased 1.9-fold, from 0.41±0.04 to 0.77±0.12 mmol/l (Table 3 and FIG. 6), and 3-β-hydroxybutyrate concentrations increased 2.2-fold. The diet lowered plasma glucose and insulin concentrations by 10% and 60%, respectively, resulting in a 64% decrease in insulin resistance (HOMA) after two weeks. Plasma triglyceride concentrations were 19% lower, but TNF-a was 17% higher after the diet (Table 3.) The diet did not alter the haematocrit, suggesting no changes in fluid homeostasis and hydration, nor did it alter plasma HbA_{1c} cholesterol, C-reactive protein or IL-6 concentrations.

Two weeks after returning to a normal diet, plasma concentrations of free fatty acids (FIG. 6) and all other metabolites (data not shown) had returned to pre-diet levels.

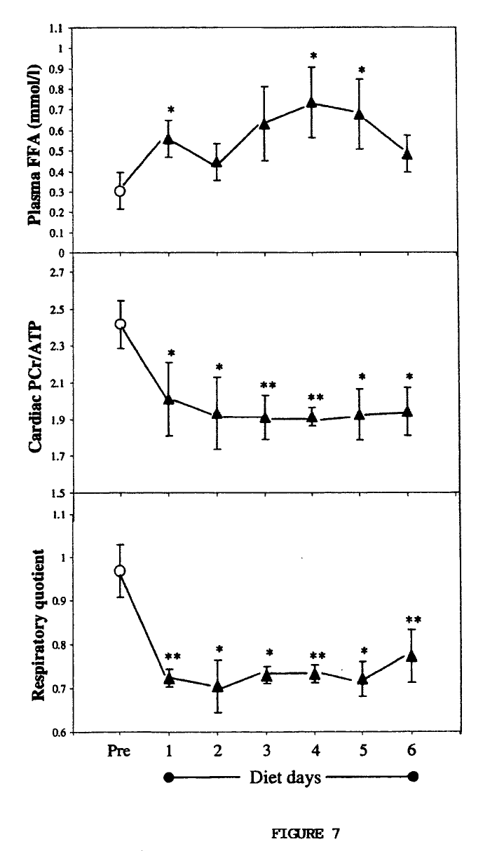

In a subgroup of subjects (n=6), fasting plasma free fatty acid concentrations, measured daily for the first 6 days of the diet, increased significantly to 0.56±0.09 mmol/l after one day of diet (FIG. 7) and remained high for the duration of the diet.

TABLE 3
Subjects characteristics and fasting blood metabolite
concentrations before and after two weeks on a high-fat, low-
carbohydrate diet

| | Before diet | two weeks on diet | |
|---|---|---|---|
| | (n = 19) | (n = 19) | p value |
| Males (%) | 10 (53) | | |
| Age (y) | 36 ± 3 | | |
| Body weight (kg) | 78.3 ± 3.9 | 75.2 ± 3.7 | <0.001 |
| BMI (kg/m²) | 26.3 ± 0.9 | 25.2 ± 0.9 | <0.001 |
| Free fatty acids (mmol/l) | 0.41 ± 0.04 | 0.77 ± 0.12 | 0.006 |
| Glucose (mmol/l) | 5.2 ± 0.2 | 4.6 ± 0.1 | 0.009 |
| HbA_{1c} (%) | 5.4 ± 0.1 | 5.4 ± 0.1 | 0.47 |
| Insulin (mU/l) | 6.1 ± 1.1 | 2.4 ± 0.4 | <0.001 |
| Insulin resistance (HOMA) | 1.44 ± 0.30 | 0.52 ± 0.10 | 0.002 |
| 3-β-hydroxybutyrate | 391 ± 137 | 843 ± 77 | 0.01 |
| (mmol/l) | | | |
| Total cholesterol (mmol/l) | 5.2 ± 0.2 | 5.7 ± 0.3 | 0.07 |
| HDL cholesterol (mmol/l) | 1.41 ± 0.05 | 1.44 ± 0.09 | 0.47 |
| Triglycerides (mmol/l) | 1.19 ± 0.15 | 0.96 ± 0.14 | 0.03 |
| C-reactive protein (mg/l) | 4.5 ± 1.04. | 0 ± 0.7 | 0.31 |
| Interleukin-6 (pg/ml) | 3.6 ± 1.2 | 0.9 ± 0.1 | 0.07 |
| Tumour necrosis factor-α | 11.5 ± 2.2 | 13.5 ± 2.1 | 0.007 |
| (pg/ml) | | | |
| Haematocrit (l/l) | 0.45 ± 0.01 | 0.44 ± 0.01 | 0.48 |

Data are presented as means ± SEM.
BMI, body mass index.

Cardiac Energetics, Respiratory Quotients and Cardiac Volumes and Function

After two weeks of high-fat, low-carbohydrate diet, cardiac PCr/ATP ratios had declined significantly, from 2.34±0.07 to 1.97±0.09, but returned to pre-diet levels two weeks after returning to a normal diet (FIG. 6). After the first day of diet, cardiac PCr/ATP was significantly lower, at 2.01±0.20, and remained low, being 1.94±0.13 after 6 days of the diet (FIG. 7). The decline in PCr/ATP was accompanied by an increase in plasma free fatty acid concentrations and a decrease in respiratory quotient, an index of the ratio of fat to carbohydrate oxidation²⁵, which fell significantly within one day of diet from 0.97±0.06 to 0.72±0.02, to remain significantly lower for at least 6 diet days (FIG. 3), indicating increased fat oxidation.

After two weeks of diet, left ventricular end-diastolic volumes were 7% smaller, whereas end-systolic volumes were not altered by the diet (Table 4). Stroke volumes and cardiac output were 11% and 8% lower, respectively, but heart rate was unchanged compared with pre-diet values. Left ventricular ejection fraction and peak ejection rate were normal, but peak filling rate was reduced after two weeks of diet, indicating diastolic dysfunction (Table 4).

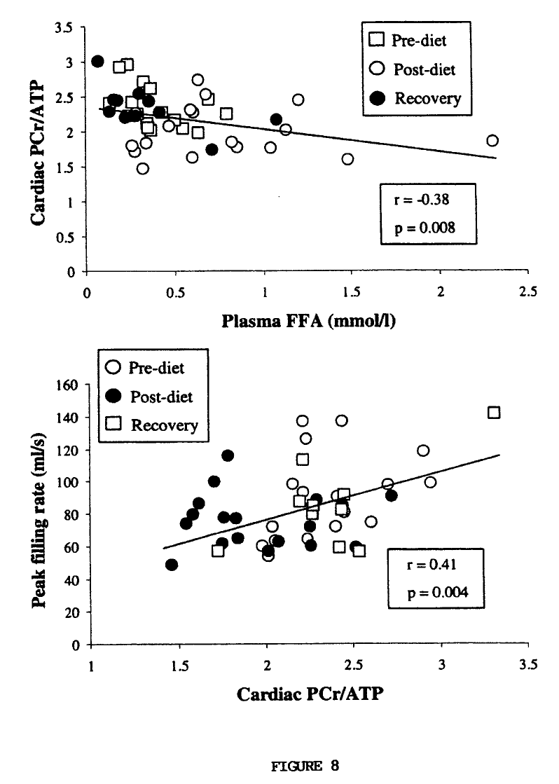

Significant negative correlations were found between cardiac PCr/ATP and plasma free fatty acid concentrations (FIG. 8) and between plasma FFA concentrations and peak filling rate (r=−0.32, p=0.03, data not shown). There was a positive correlation between peak filling rate and cardiac PCr/ATP (FIG. 8). Thus, plasma free fatty acid concentrations are closely associated with cardiac energetics and diastolic function.

TABLE 4
Left ventricular cardiac volumes and function
before and after two weeks on a high-fat, low-
carbohydrate diet

| | Before diet | two weeks on diet | |
|---|---|---|---|
| | (n = 19) | (n = 19) | p value |
| End-diastolic volume (ml) | 114 ± 6 | 106 ± 6 | 0.001 |
| End-systolic volume (ml) | 32 ± 2 | 30 ± 3 | 0.15 |
| Stroke volume (ml) | 83 ± 4 | 74 ± 3 | 0.001 |
| Cardiac output (l/min) | 5.3 ± 0.3 | 4.9 ± 0.2 | 0.02 |
| Heart rate (bpm) | 65 ± 2 | 68 ± 2 | 0.06 |
| Ejection fraction (%) | 73 ± 1 | 72 ± 1 | 0.27 |
| Peak ejection rate (ml/s) | 84 ± 6 | 74 ± 5 | 0.08 |
| Peak filling rate (ml/s) | 90 ± 6 | 75 ± 4 | 0.01 |

Data are presented as means ± SEM.

### REFERENCE

1. Schernthaner G. Cardiovascular mortality and morbidity in type-2 diabetes mellitus. Diabetes Res Clin Prac 1996;31:S3-S13.
2. Taegtmeyer H, McNulty P, Young M E. Adaptation and maladaptation of the heart in diabetes: Part I. General concepts. Circulation. 2002;105:1727-1733.
3. Iribarren C, Karter A J, Go A S, et al. Glycemic control and heart failure among adult patients with diabetes. Circulation. 2001;103:2668-2673.
4. Rodrigues B, Cam M C, McNeill J H. Metabolic disturbances in diabetic cardiomyopathy. Mol Cell Biochem. 1998; 180:53-57.
5. Chatham J C, Seymour A-M L. Cardiac carbohydrate metabolism in Zucker diabetic fatty rats. Cardiovasc Res. 2002;55:104-112.
6. Sidell R J, Cole M A, Draper N J, et al. Thiazolidinedione treatment normalizes insulin resistance and ischemic injury in the Zucker fatty rat heart. Diabetes. 2002;51:1110-1117.
7. Stanley W C, Lopaschuk G D, McCormack J G. Regulation of energy substrate metabolism in the diabetic heart. Cardiovasc Res. 1997;34:25-33.
8. Tune J D, Yeh C, Setty S, et al. Coronary blood flow control is impaired at rest and during exercise in conscious diabetic dogs. Basic Res Cardiol. 2002;97:248-257.
9. Meyer C, Schwaiger M. Myocardial blood flow and glucose metabolism in diabetes mellitus. Am J Cardiol. 1997;80:94A-101A.
10. lozzo P, Chareonthaitawee P, Rimoldi O, et al. Mismatch between insulin-mediated glucose uptake and blood flow in the heart of patients with Type II diabetes. Diabetologia. 2002;45:1404-1409.
11. Baron A D, Laakso M, Brechtel G, et al. Mechanism of insulin resistance in insulin-dependent diabetes mellitus: A major role for reduced skeletal muscle blood flow. J Clin Endocrinol Metab. 1991;73:637-643.
12. Demir I, Ermis C, Altunbas H, et al. Serum HbA1c levels and exercise capacity in diabetic patients. Jon Heart J. 2001:42-607-616.
13. Estacio R O, Regensteiner J G, Wolfel E E, et al. The association between diabetic complications and exercise capacity in NIDDM patients. Diabetes Care. 1998;21:291-295.
14. Crilley J G, Boehm E A, Rajagopalan B, et al. Magnetic resonance spectroscopy evidence of abnormal cardiac energetics in Xp21 muscular dystrophy. J Am Coll Cardiol. 2000;36:1953-1958.
15. Lodi R, Kemp G J, Muntoni F, et al. Reduced cytosolic acidification during exercise suggests defective glycolytic activity in skeletal muscle of patients with Becker muscular dystrophy. An in vivo ³¹P magnetic resonance spectroscopy study. Brain. 1999; 122:121-130.
16. Veech R L, Lawson J W R, Cornell N W, et al. Cytosolic phosphorylation potential. J Biol Chem. 1979;254:6538-6547.
17. Madsen P L, Secher N H. Near-infrared oximetry of the brain. Prog Neurobiol. 1999;58:541-560.
18. Neubauer S, Horn M, Cramer M, et al. Myocardial phosphocreatine-to-ATP ratio is a predictor of mortality in patients with dilated cardiomyopathy. Circulation. 1997;96:2190-2196.
19. Boehm E A, Jones B E, Radda G K, et al. Increased uncoupling proteins and decreased efficiency in the palmitate-perfused hyperthyroid rat heart. Am J Physiol. 2001;280:H977-H983.
20. Chatham J C, Gao Z P, Bonen A, et al. Preferential inhibition of lactate oxidation relative to glucose oxidation in the rat heart following diabetes. Cardiovasc Res. 1999;43:96-106.
21. Libby P, Plutzky J. Diabetic macrovascular disease. The glucose paradox? Circulation. 2002;106:2760-2763.
22. Mahler R J, Adler M L. Type 2 diabetes mellitus: update on diagnosis, pathophysiology, and treatment. J Clin Endocrinol Metab. 1999;84:1165-1171.
23. Komiyama T, Shigematsu H, Yasuhara H, et al. Near-infrared spectroscopy grades the severity of intermittent claudication in diabetes more accurately than ankle pressure measurement. Br J Surg. 2000:87:459-466.
24. Matthews D R, Hosker J P, Rudenski A S, Naylor B A, Treacher D F, Turner R C. Homeostasis model assessment: insulin resistance and beta-cell function from fasting plasma glucose and insulin concentrations in man. Diabetologia 1985; 28:412-419.
25. Toubro S, Sorenson T, Hindsberger C, Christensen N, Astrup A Twenty-four-hour respiratory quotient: the role of diet and familial resemblance. J Clin Endocrinol Metab 1998; 83:2758-2764.
26. Paolisso G, Tagliamonte M R, Rizzo M R, et al. Prognostic importance of insulin-mediated glucose uptake in aged patients with congestive heart failure secondary to mitral and/or aortic valve disease. Am J of Cardiol 1999; 83:1338-1344.
27. Rodrigues B, Cam M C, McNeill J H. Metabolic disturbances in diabetic cardiomyopathy. Mol Cell Biochem 1998; 180:53-57.
28. Komiyama N, Saito T, Hosaka Y, et al. Effects of a 4-week 70% high carbohydrate/15% low fat diet on glucose tolerance and on lipid profiles. Diabetes Res Clin Pract 2004; 64:11-8.
29. Perez-Jimenez F, Lopez-Miranda J, Pinillos M, et al. A mediterranean and a high-carbohydrate diet improve glucose metabolism in healthy young persons. Diabetologia 2001; 44:2038-2043.
30. Scheuermann-Freestone M, Radda G, Neubauer S, Clarke K. Abnormal cardiac and skeletal muscle function and energetics correlated with insulin resistance in heart failure. Circulation 2004; 110:III-335.
31. Scheuermann-Freestone M, Madsen P L, Manners D, et al. Abnormal cardiac and skeletal muscle energy metabolism in patients with type 2 diabetes. Circulation 2003; 107:3040-3046.

1. A method for preventing and treating muscle fatigue comprising the step of administering to a subject a compound that reduces concentrations of free fatty acids circulating in the plasma of the subject, wherein the muscle is selected from the group consisting of cardiac and skeletal muscle.

2. The method according to claim 1 wherein the concentrations of free fatty acids in the plasma of the subject is due to a disorder associated with mitochondrial dysfunction.

3. The method according to claim 1 wherein the concentrations of free fatty acids in the plasma of the subject is due to impairment of muscle function.

4. The method according to claim 1 wherein the subject has diabetes.

5. The method according to claim 4 wherein the diabetes is type 2 diabetes.

6. The method according to claim 1 wherein the compound is selected from the group consisting of ketone bodies, nicotinic acid, salicyclic acid, thiazolidine diones, and fibrates.

7. The method according to claim 1 wherein the compound is in the form selected from the group consisting a food supplement and a liquid composition.

8. A composition for rehydrating a subject, the composition comprising water, a sugar carbohydrate, and a compound that reduces concentrations of free fatty acids circulating in the plasma of the subject, wherein rehydrating the subject is preformed at a period selected from the group consisting of during an exercise period and following an exercise period.

9. The composition according to claim 8 wherein the sugar carbohydrate is glucose.

10. A method for monitoring cardiac muscle function in a subject, the method comprising the steps of: i) measuring concentrations of free fatty acids in a blood plasma sample from a fasted subject and ii) quantifying the result with levels of free fatty acids sampled from a control subject.

11. The method according to claim 10, wherein cardiac muscle function shows impairment at a concentration of free fatty acids of greater that 0.5 mM in a blood plasma sample from a subject undertaking a diet.

12. The method according to claim 11, wherein the subject is undertaking a high-fat low-carbohydrate diet.

13. The method according to claim 1, wherein the subject is healthy and non-obese.

14. A device for monitoring cardiac muscle function, the device comprising means for measuring the concentrations of free fatty acids in a plasma sample.

15. The device according to claim 14, wherein the device is a hand-held device and is shaped, sized, and adapted for use in the hand of an individual.

16. The device according to claim 14, wherein the device further comprises an electrochemical cell.
